(19) United States
(12) Patent Application Publication
(10) Pub. No.: US 2026/0102381 A1
(43) Pub. Date: Apr. 16, 2026

(54) Muscarinic Agent Combinations

(71) Applicant: Syremis Therapeutics, Ltd., Petach Tikva (IL)

(72) Inventor: Hafiz Mawasi, Petach Tikva (IL)

(73) Assignee: Syremis Therapeutics, Ltd., Petach Tikva (IL)

(21) Appl. No.: 19/419,365

(22) Filed: Dec. 15, 2025

Related U.S. Application Data
(63) Continuation of application No. PCT/IB2025/057155, filed on Jul. 15, 2025.
(60) Provisional application No. 63/672,136, filed on Jul. 16, 2024.

(51) Int. Cl. A61K 31/439; A61K 9/00; A61K 31/40; A61P 1/02

(52) U.S. Cl. CPC A61K 31/439; A61K 9/0053; A61K 31/40; A61P 1/02

(57) ABSTRACT

The invention relates to a combination of sabcomeline, or a pharmaceutically acceptable salt thereof, and a long-acting non-Central Nervous System penetrant muscarinic antagonist. The invention further relates to pharmaceutical compositions comprising the combination of sabcomeline, or a pharmaceutically acceptable salt thereof, and a long-acting non-Central Nervous System penetrant muscarinic antagonist and uses thereof.

### CROSS-REFERENCE TO RELATED APPLICATIONS

This application is a continuation of International Patent Application No. PCT/IB2025/057155 filed on Jul. 15, 2025 which claims the benefits of U.S. Provisional Application Ser. No. 63/672,136, filed on Jul. 16, 2024, the disclosure of which is incorporated herein by reference in its entirety.

### BACKGROUND TO THE INVENTION

Sabcomeline is an M1/M4 muscarinic agonist. Although sabcomeline hydrochloride was found to be safe in the elderly population when dosed twice daily at 75 μg dose (Kumar et al 1997), its development was halted in the late 1990s after the product failed in Phase III studies in the symptomatic treatment of Alzheimer's disease.

Methods of manufacturing the compound and its oxalate salt are described in EP392803. WO9704750 describes various immediate release formulations of sabcomeline monohydrochloride, while WO9810762 and WO9945924 both describe various controlled release formulations of the monohydrochloride salt and WO9804258 describes transdermal compositions. None of these references contemplate the product being used in combination with additional active agents.

WO2011011060 describes the combination of one or more muscarinic agonists and one or more muscarinic antagonists for treatment of diseases that are ameliorated by activation of muscarinic receptors. Due to the known failure (Maral et al. Neurology. 38:606. 1988) of an earlier attempt to mitigate the side-effects of a muscarinic agonist (RS-86) with an antagonist (glycopyrrolate), the reference warns about the extremely difficult task of predicting, a priori, and without any in vivo evidence, whether any particular agonist/antagonist combination would be both safe and efficacious. As such, the reference relies on an in-silico analysis to propose a variety of possible combination products that might possibly be of use in decreasing the side-effects associated with the agonist and provides 75 distinct scoring criteria for any antagonist considered. The reference does not elaborate on the scoring of these considerations sufficiently to be able to actually rank any combination. Amongst the 15 different combinations that the reference proposes are sabcomeline and trospium chloride, sabcomeline and controlled release trospium chloride, and sabcomeline and solifenacin.

More recently, xanomeline, a muscarinic acetyl choline receptor agonist, in combination with trospium (30 mg), was associated with clinically meaningful improvement in schizophrenia patients experiencing acute psychosis.

Cooper et al (1996) reported that single doses of 125-150 μg of sabcomeline hydrochloride were found by healthy volunteers to be intolerable due to sweating despite the incidence of other side effects being low. In humans, sweating and salivation are both secretory processes controlled by the sympathetic nervous system and are induced by activation of the M3 muscarinic receptor. Most muscarinic antagonists, including trospium, are approved for treating urinary incontinence. Some are also approved in inhaled form as bronchodilators. Antagonism of the M3 receptor is known to reduce both sweating and salivation and remains a side-effect of some muscarinic antagonists. Glycopyrrolate is the only muscarinic antagonist specifically approved to treat both excess sweating and excess salivation.

Gavaldà et al (2014) demonstrated a correlation between M3 receptor antagonist dissociation times and their duration of activity as bronchodilators. In their study, they demonstrated that ipratroprium was both a short-acting bronchodilator and also had a short dissociation time from the M3 receptor. In contrast, aclidinium, glycopyrronium and tiotropium were each shown to have both longer dissociation times on the receptor and to be longer-acting bronchodilators. Oki et al (2006) demonstrated that solifenacin had both a short dissociation time on the M3 receptor and was also less effective and with a shorter duration of action than oxybutynin in a rat pilocarpine-induced salivation model.

There remains a need for safe and effective methods of treating schizophrenia and other conditions.

### SUMMARY OF THE INVENTION

The invention is directed to the combination of sabcomeline, or a pharmaceutically acceptable salt thereof, and a long-acting non-Central Nervous System (CNS) penetrant muscarinic antagonist.

The present invention also relates to a fixed dose combination of sabcomeline, or a pharmaceutically acceptable salt thereof, and a long-acting non-CNS penetrant muscarinic antagonist.

### DETAILED DESCRIPTION OF THE INVENTION

The present inventions may be understood more readily by reference to the following detailed description taken in connection with any accompanying figures and examples, which form a part of this disclosure. It is to be understood that these inventions are not limited to the specific products, methods, conditions or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a”, “an”, and “the” include the plural reference, and reference to a particular numerical value includes at least that particular value, unless the context clearly indicates otherwise. Thus, for example, a reference to “a particle” is a reference to one or more of such particles and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about”, it will be understood that the particular value forms another embodiment. As used herein, “about X” (where X is a numerical value) preferably refers to 10% of the recited value, inclusive. For example, the phrase “about 8” preferably refers to a value of 7.2 to 8.8, inclusive; as another example, the phrase “about 8%” preferably refers to a value of 7.2% to 8.8%, inclusive. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included”. The phrase “at least about x” is intended to embrace both “about x” and “at least x”. It is also understood that where a parameter range is provided, all integers within that range, and tenths thereof, are also provided by the invention. For example, “2-5 hours” includes 2 hours, 2.1 hours, 2.2 hours, 2.3 hours etc., up to 5 hours.

The present invention is directed to pharmaceutical compositions comprising a fixed dose combination of sabcomeline, or a pharmaceutically acceptable salt thereof, and a long-acting muscarinic antagonist, and wherein said muscarinic antagonist does not substantially cross the blood-brain-barrier.

As used herein, the term “pharmaceutical composition” refers to a composition comprising at least one active ingredient and at least one pharmaceutically acceptable excipient.

As used here, the term “dosage form” refers to a drug product comprising at least one active ingredient.

As used herein, the term “sabcomeline” refers to the compound (αZ,3R)-α-(Methoxyimino)-1-azabicyclo[2.2.2]octane-3-acetonitrile and pharmaceutically acceptable salts thereof.

As used herein, the term “pharmaceutically acceptable salts” can be used interchangeably with the term “salts” and refers to salts prepared from relatively non-toxic acids or bases including inorganic acids and bases and organic acids and bases, including, for example, those contained in compositions of the present invention. Suitable non-toxic acids may include inorganic and organic acids such as hydrochloric, hydrobromic, phosphoric, acetic, fumaric, salicylic, citric, lactic, mandelic, tartaric, oxalic, methanesulphonic and the like. In one embodiment of the invention, the sabcomeline, or a pharmaceutically acceptable salt thereof, is the hydrochloride salt of sabcomeline, i.e., sabcomeline monohydrochloride.

As used herein, the term “muscarinic antagonists” shall refer to molecules that behave as antagonists, partial antagonists, competitive antagonists, non-competitive antagonists, uncompetitive antagonists, silent antagonists, inverse agonists, reversible antagonists, physiological antagonists, irreversible antagonists, inhibitors, reversible inhibitors, irreversible inhibitors, negative allosteric modulators, allosteric antagonists at one or more muscarinic receptors or to molecules that decrease the activity or signalling of muscarinic receptors through direct or indirect means. Methods of determining whether a molecule is a muscarinic antagonist are known in the art. Examples of muscarinic antagonists include aceclidine, aclidinium, acotiamide, alvameline, ambutonium, anagyrine, anethole trithione, anisodamine, arborine, atropine, atropine methyl bromide, batefenterol, beperidium, benzetimide, benztropine, biperiden, blarcamesine, camylofin, cevimeline, clozapine, cimetropium, cisapride, clidinium, cyclobuxine D, cyclodrine, cyclopentolate, darenzepine, darifenacin, darotropium, desfesoterodine, dexetimide, dicyclomine, diphenidol, diphemanil, dimethindene, dextro-mequitamium, dronedarone, ebeinone, elucaine, emraclidine, esoxybutynin, espartropate, fesoterodine, flavoxate, gallamine, glycopyrronium, heliosupine n-oxide, homatropine, imidafenacin, ipratropium, irsogladine, liriodenine, l-hyoscyamine, mazaticol, mequitamiom, methacholine chloride, methantheline, methoctramine tetrahydrochloride, methylbenactyzium, methscopolamine, metixene, n-butylscopalmine, n-methylatropine, navafenterol, nuvenzepine, octamylamine, octylonium, otenzepad, oxitropium, oxotremorine, oxybutynin, peimisine, penehyclidine, phenglutarimid, pirenzepine, pirmenol, piperidolate, pirenzepine, promethazine, propantheline, propiverine, prifinium, rapacuronium, revatropate, revefenacin, rispenzepine, scopolamine, secoverine, sintropium, sofpironium, solifenacin, tarafenacin, telenzepine, temivirine, terodiline, timepidium, tiotropium, tolterodine, tropenziline, tropicamide, trospium, trihexyphenidyl, zamifenacin, 4-DAMP and umeclidinium and pharmaceutically acceptable salts thereof.

As used herein, the term “muscarinic receptors” refers to G-protein linked receptors that bind the neurotransmitter acetylcholine. To date, five subtypes of muscarinic receptor have been identified. “M1” means the subtype one muscarinic receptor. “M2” means the subtype two muscarinic receptor. “M3” means the subtype three muscarinic receptor. “M4” means the subtype four muscarinic receptor. “M5” means the subtype five muscarinic receptor.

As used herein, the term “long-acting muscarinic antagonist” shall refer to a muscarinic antagonist having an M3 receptor dissociation half-life of at least 4 hours according to the methodology laid out in Gavaldà et al (2014). Examples of long-acting muscarinic antagonists include aclidinium, glycopyrronium, tiotropium and their pharmaceutically acceptable salts. Trospium and its pharmaceutically acceptable salts is not considered to be a long-acting muscarinic antagonist.

In one embodiment of the invention, the muscarinic antagonist has an M3 dissociation half life of at least 4 hours according to the methodology laid out in Gavaldà et al (2014). In another embodiment of the invention, the muscarinic antagonist has an M3 dissociation half life of at least 4 hours, at least 5 hours, at least 6 hours, at least 7 hours, at least 8 hours, at least 9 hours, at least 10 hours, at least 11 hours or at least 12 hours according to the methodology laid out in Gavaldà et al (2014).

As used herein, the term “does not substantially cross the blood-brain-barrier” can be used interchangeably with the term “non-CNS penetrant” and means that most, almost all, or all of the amount of antagonist administered to the subject does not cross the blood brain barrier. For example, less than 25 percent, less than 20 percent, less than 15 percent, less than 10 percent, less than 5, less than 3 percent, or less than 1 percent of the amount of antagonist administered does not cross the blood brain barrier. Methods of determining whether a molecule crosses the blood brain barrier are known in the art. Examples of non-CNS penetrant muscarinic antagonists include aclidinium, glycopyrronium, oxitropium, propiverine, tiotropium, trospium and pharmaceutically acceptable salts thereof and examples of CNS penetrant muscarinic antagonists, i.e., antagonists that when administered to the subject do cross the blood brain barrier, include oxybutynin, solifenacin and tolterodine. In one embodiment of the invention, the non-CNS penetrant muscarinic antagonist is glycopyrronium or a pharmaceutically acceptable salt thereof. In a preferred embodiment of the invention, the non-CNS penetrant muscarinic antagonist is glycopyrronium halide salt such as the hydrobromide, i.e., glycopyrrolate. In another preferred embodiment of the invention, the non-CNS penetrant muscarinic antagonist is glycopyrronium tosylate.

In one embodiment of the invention, sabcomeline may be taken sequentially with the long-acting non-CNS penetrant muscarinic antagonist. In another embodiment of the invention, sabcomeline may be taken concurrently with the long-acting non-CNS penetrant muscarinic antagonist. In a preferred embodiment of the invention, sabcomeline and the long-acting non-CNS penetrant muscarinic antagonist are formulated to be contained in the same dosage form or dosage vehicle. In another embodiment of the invention, sabcomeline and the long-acting non-CNS penetrant muscarinic antagonist are formulated to be in separate dosage forms or dosage vehicles.

In one embodiment of the invention, sabcomeline and the long-acting non-CNS penetrant muscarinic antagonist are in different dosage forms or dosage vehicles. In a preferred embodiment of the invention, sabcomeline and the long-acting non-CNS penetrant muscarinic antagonist are in the same dosage form or dosage vehicles.

The dosage forms may include one or more pharmaceutically-acceptable carriers. The dosage forms may also include one or more pharmaceutically-acceptable salts. The dosage forms may be administered orally using tablets, troches, liquids, emulsions, suspensions, drops, capsules, caplets or gel caps and other methods of oral administration known to one skilled in the art. Dosage forms may also be administered parentally. Other routes of administration include but are not limited to: topical, transdermal, nasal, ocular, rectal, sublingual, inhalation, and vaginal. For parenteral administration, the dosage forms may be delivered in injections, solutions, emulsions or suspensions or by other methods known to one skilled in the art. For topical and transdermal administration, the dosage forms may be delivered in a cream, gel, ointment, spray, suspension, emulsion, foam, or patch or by other methods known to one skilled in the art. For nasal administration, the dosage forms may be delivered by sprays, drops, emulsions, foams, creams, ointments or other methods known to one skilled in the art. For nasal administration, formulations for inhalation may be prepared as an aerosol, either a solution aerosol in which the active agent is solubilized in a carrier, such as a propellant, or a dispersion aerosol, in which the active agent is suspended or dispersed throughout a carrier and an optional solvent. For ocular administration, dosage forms may be delivered in drops, sprays, injections, solutions, emulsions, suspensions, or ointments, or by other methods known to one skilled in the art. For rectal administration, dosage forms may be delivered using suppositories, enemas, creams, foams, gels, or ointments or by other methods known to one skilled in the art. For sublingual administration, dosage forms may be delivered in tablets, troches, liquids, emulsions, suspensions, drops, capsules, caplets or gel caps and by other methods of oral administration known to one skilled in the art. For administration by inhalation, dosage forms may be delivered in vapor, mist, powder, aerosol, or nebulized form, or by other methods known to one skilled in the art. For vaginal administration, dosage forms may be delivered in solutions, emulsions, suspensions, ointments, gels, foams, or vaginal rings or by other methods known to one skilled in the art.

In some aspects of the invention, pharmaceutical compositions of the disclosure include sabcomeline, i.e., sabcomeline as a free base. In other aspects of the invention, pharmaceutical compositions of the disclosure include pharmaceutically acceptable salts of sabcomeline such as described in International Patent Application No. PCT/IB2025/056914 filed on Jul. 8, 2025 which is incorporated herein by reference. As used herein and unless specifically stated otherwise, amounts of sabcomeline present in the pharmaceutical compositions of the disclosure refer to amounts of sabcomeline free base. For example, in those aspects wherein the pharmaceutical composition comprises sabcomeline free base, “10 mg of sabcomeline” refers to 10 mg of the sabcomeline free base in the pharmaceutical compositions. In aspects wherein the oral dosage form comprises a pharmaceutically acceptable salt of sabcomeline, such as sabcomeline hydrochloride, “10 mg of sabcomeline” refers to 10 mg sabcomeline free base, based on 11.89 mg of sabcomeline hydrochloride in the pharmaceutical composition.

In some aspects of the invention, pharmaceutical compositions of the disclosure include a non-CNS penetrant muscarinic antagonist as a free base. In other aspects of the invention, pharmaceutical compositions of the disclosure include pharmaceutically acceptable salts of a non-CNS penetrant muscarinic antagonist. As used herein, amounts of a non-CNS penetrant muscarinic antagonist present in the pharmaceutical compositions of the disclosure refer to the amounts of non-CNS penetrant muscarinic antagonist whether present as free base or as a pharmaceutically acceptable salt. For example, in those aspects wherein the pharmaceutical composition comprises non-CNS penetrant muscarinic antagonist free base, “10 mg of non-CNS penetrant muscarinic antagonist” refers to 10 mg of the non-CNS penetrant muscarinic antagonist free base in the pharmaceutical compositions. In aspects wherein the oral dosage form comprises a pharmaceutically acceptable salt of a non-CNS penetrant muscarinic antagonist, such as non-CNS penetrant muscarinic antagonist hydrobromide, “10 mg of non-CNS penetrant muscarinic antagonist” refers to 10 mg of a non-CNS penetrant muscarinic antagonist hydrobromide salt as present in the pharmaceutical composition unless specifically stated otherwise.

In one embodiment of the invention, the pharmaceutical composition comprises between about 25 μg and about 60 mg of sabcomeline. In one embodiment of the invention, the pharmaceutical composition comprises about 25 μg, about 50 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, about 700 μg, about 725 μg, about 750 μg, about 775 μg, about 800 μg, about 825 μg, about 850 μg, about 875 μg, about 900 μg, about 925 μg, about 950 μg, about 975 μg, about 1 mg, about 1.25 mg, about 1.5 mg, about 1.75 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg or about 60 mg of sabcomeline.

In one embodiment of the invention, the pharmaceutical composition comprises an oral dosage form comprising between about 25 μg and about 1 mg of sabcomeline. In one embodiment of the invention, the pharmaceutical composition comprises an oral dosage form comprising about 25 μg, about 50 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, about 700 μg, about 725 μg, about 750 μg, about 775 μg, about 800 μg, about 825 μg, about 850 μg, about 875 μg, about 900 μg, about 925 μg, about 950 μg, about 975 μg or about 1 mg of sabcomeline.

In one embodiment of the invention, the pharmaceutical composition comprises a parenteral dosage form comprising between about 25 μg and about 60 mg of sabcomeline. In one embodiment of the invention, the pharmaceutical composition comprises a parenteral dosage form comprising about 25 μg, about 50 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, about 700 μg, about 725 μg, about 750 μg, about 775 μg, about 800 μg, about 825 μg, about 850 μg, about 875 μg, about 900 μg, about 925 μg, about 950 μg, about 975 μg, about 1 mg, about 1.25 mg, about 1.5 mg, about 1.75 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg or about 60 mg of sabcomeline.

In one embodiment of the invention, the pharmaceutical composition provides a daily dose of between about 25 μg and about 1 mg of sabcomeline following administration to a subject or patient. In one embodiment of the invention, the pharmaceutical composition provides a daily dose of about 25 μg, about 50 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, about 700 μg, about 725 μg, about 750 μg, about 775 μg, about 800 μg, about 825 μg, about 850 μg, about 875 μg, about 900 μg, about 925 μg, about 950 μg, about 975 μg or about 1 mg of sabcomeline.

In one embodiment of the invention, the pharmaceutical composition comprises between about 0.01 and about 600 mg of a long-acting non-CNS penetrant muscarinic antagonist. In one embodiment of the invention, the pharmaceutical composition comprises about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.125 mg, about 0.2 mg, about 0.25 mg, about 0.3 mg, about 0.4 mg, about 0.5 mg, about 0.6 mg, about 0.7 mg, about 0.75 mg, about 0.8 mg, about 0.9 mg, about 1 mg, about 1.5 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 17.5 mg, about 20 mg, about 22.5 mg, about 25 mg, about 30 mg, about 35 mg, about 40 mg, about 50 mg, about 60 mg, about 70 mg, about 80 mg, about 90 mg, about 100 mg, about 120 mg, about 150 mg, about 180 mg, about 200 mg, about 210 mg, about 240 mg, about 250 mg, about 280 mg, about 300 mg, about 330 mg, about 350 mg, about 360 mg, about 390 mg, about 400 mg, about 420 mg, about 450 mg, about 480 mg, about 500 mg, about 510 mg, about 540 mg, about 550 mg, about 580 mg or about 600 mg of a long-acting non-CNS penetrant muscarinic antagonist.

In one embodiment of the invention, the pharmaceutical composition comprises an oral dosage form comprising between about 0.01 and about 20 mg of a long-acting non-CNS penetrant muscarinic antagonist. In one embodiment of the invention, the pharmaceutical composition comprises an oral dosage form comprising about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.125 mg, about 0.2 mg, about 0.25 mg, about 0.3 mg, about 0.4 mg, about 0.5 mg, about 0.6 mg, about 0.7 mg, about 0.75 mg, about 0.8 mg, about 0.9 mg, about 1 mg, about 1.5 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 17.5 mg or about 20 mg of a long-acting non-CNS penetrant muscarinic antagonist.

In one embodiment of the invention, the pharmaceutical composition comprises a parenteral dosage form comprising between about 0.01 and about 600 mg of a long-acting non-CNS penetrant muscarinic antagonist. In one embodiment of the invention, the pharmaceutical composition comprises a parenteral dosage form comprising about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.125 mg, about 0.2 mg, about 0.25 mg, about 0.3 mg, about 0.4 mg, about 0.5 mg, about 0.6 mg, about 0.7 mg, about 0.75 mg, about 0.8 mg, about 0.9 mg, about 1 mg, about 1.5 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 17.5 mg, about 20 mg, about 22.5 mg, about 25 mg, about 30 mg, about 35 mg, about 40 mg, about 50 mg, about 60 mg, about 70 mg, about 80 mg, about 90 mg, about 100 mg, about 120 mg, about 150 mg, about 180 mg, about 200 mg, about 210 mg, about 240 mg, about 250 mg, about 280 mg, about 300 mg, about 330 mg, about 350 mg, about 360 mg, about 390 mg, about 400 mg, about 420 mg, about 450 mg, about 480 mg, about 500 mg, about 510 mg, about 540 mg, about 550 mg, about 580 mg or about 600 mg of a long-acting non-CNS penetrant muscarinic antagonist.

In one embodiment of the invention, the pharmaceutical composition provides a daily dose of between about 0.01 and about 20 mg of a long-acting non-CNS penetrant muscarinic antagonist following administration to a subject or patient. In one embodiment of the invention, the pharmaceutical composition provides a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.125 mg, about 0.2 mg, about 0.25 mg, about 0.3 mg, about 0.4 mg, about 0.5 mg, about 0.6 mg, about 0.7 mg, about 0.75 mg, about 0.8 mg, about 0.9 mg, about 1 mg, about 1.5 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 17.5 mg or about 20 mg of a long-acting non-CNS penetrant muscarinic antagonist following administration to a subject or patient.

The present invention is also directed to pharmaceutical compositions comprising the fixed dose combination of sabcomeline, or a pharmaceutically acceptable salt thereof, and a long-acting muscarinic antagonist, wherein said long-acting muscarinic antagonist does not substantially cross the blood-brain-barrier.

In one embodiment of the invention, the daily dose of the long-acting non-CNS penetrant muscarinic antagonist is no more than about 20 mg, no more than about 19 mg, no more than about 18 mg, no more than about 17 mg, no more than about 16 mg, no more than about 15 mg, no more than about 14 mg, no more than about 13 mg, no more than about 12 mg, no more than about 11 mg, no more than about 10 mg, no more than about 9 mg, no more than about 8 mg, no more than about 7.5 mg, no more than about 7 mg, no more than about 6.66 mg, no more than about 6 mg, no more than about 5 mg, no more than about 4.5 mg, no more than about 4 mg, no more than about 3.5 mg, no more than about 3.33 mg, no more than about 3 mg, no more than about 2.75 mg, no more than about 2.5 mg, no more than about 2.25 mg, no more than about 2 mg, no more than about 1.75 mg, no more than about 1.5 mg, no more than about 1.25 mg, no more than about 1 mg, no more than about 0.75 mg, no more than about 0.66 mg, no more than about 0.5 mg, no more than about 0.33 mg, no more than about 0.25 mg, no more than about 0.2 mg, no more than about 0.1 mg, no more than about 0.05 mg or no more than about 0.033 mg. In a preferred embodiment of the invention, the daily dose of the long-acting non-CNS penetrant muscarinic antagonist is no more than about 20 mg. In another preferred embodiment of the invention, the daily dose of the long-acting non-CNS penetrant muscarinic antagonist is no more than about 15 mg. In another preferred embodiment of the invention, the daily dose of the long-acting non-CNS penetrant muscarinic antagonist is no more than about 10 mg.

In one embodiment of the invention, the pharmaceutical composition comprises sabcomeline, or a pharmaceutically acceptable salt thereof, and glycopyrronium, of a pharmaceutically acceptable salt thereof. In a preferred embodiment, the glycopyrronium is glycopyrrolate.

The present invention is also directed to methods for treating a disorder ameliorated by activating muscarinic receptors in a subject in need thereof, comprising administering to a patient in need thereof any pharmaceutical composition described herein. In certain embodiments, the subject is a human. In certain embodiments, the disorder is selected from schizophrenia, Alzheimer's disease, Parkinson's disease, depression, movement disorders, pain, drug addiction, tauopathy, and synucleinopathy.

In one embodiment of the invention, sabcomeline is combined with a long-acting non-CNS penetrant muscarinic antagonist to treat muscarinic disorders, ameliorating symptoms in response to muscarinic activation by sabcomeline in living tissues found outside the brain. In an embodiment of the invention, such diseases or disorders include schizophrenia and diseases related to schizophrenia, cognitive disorders in neurodegenerative diseases such as Alzheimer's, and pain such as nociceptive pain or neuropathic pain. The combination of sabcomeline and a long-acting non-CNS penetrant muscarinic antagonist is a safer method for treating those diseases shown to be responsive to activation of muscarinic receptors.

As used herein, the term “muscarinic disorders” refers to any disease or condition ameliorated by activating the muscarinic system. Such diseases include ones in which direct activation of muscarinic receptors themselves or inhibition of cholinesterase enzymes has produced a therapeutic effect.

As used herein, the term “diseases related to schizophrenia” and the term “disorders related to schizophrenia” include, but are not limited to, schizo-affective disorder, psychosis, delusional disorders, psychosis associated with Alzheimer's disease, psychosis associated with Parkinson's disease, psychotic depression, bipolar disorder, bipolar with psychosis, Huntington's disease, Lewy Body dementia, or any other disease with psychotic features.

As used herein, the term “cognitive disorders” refers to diseases or disorders marked by cognitive deficit (e.g., having abnormal working memory, problem solving abilities, etc.). Diseases include but are not limited to Alzheimer's disease, Parkinson's Disease, dementia (including, but not limited to, AIDS-related dementia, vascular dementia, age-related dementia, dementia associated with Lewy bodies and idiopathic dementia), Pick's disease, tauopathies, synucleinopathies, confusion, cognitive deficit associated with fatigue, learning disorders, traumatic brain injury, autism, age-related cognitive decline, and Cushing's Disease, a cognitive impairment associated with autoimmune diseases.

As used herein, the term “pain” refers to physical suffering or discomfort caused by illness or injury. Pain is a subjective experience and the perception of pain is performed parts of the central nervous system (CNS). Usually noxious (peripheral) stimuli are transmitted to the CNS beforehand, but pain is not always associated with nociception. A broad variety of clinical pain exists, derived from different underlying pathophysiological mechanisms and needing different treatment approaches. Three major types of clinical pain have been characterized: acute pain, chronic pain, and neuropathic pain.

Acute clinical pain may result, for example, from inflammation or soft tissue injury. This type of pain is adaptive and has the biologically relevant function of warning and enabling healing and repair of an already damaged body part to occur undisturbed. A protective function is achieved by making the injured or inflamed area and surrounding tissue hypersensitive to all stimuli so that contact with any external stimulus can be avoided. The neuronal mechanisms underlying this type of clinical pain are well understood and pharmacological control of acute clinical pain is available and effective, for example by means of nonsteroidal anti-inflammatory drugs (NSAIDs) up to opioids depending on type and extent of the sensation of pain.

Chronic clinical pain appears as sustained sensory abnormalities resulting from an ongoing peripheral pathology such as cancer or chronic inflammation (e.g., arthritis) or it can be independent of such initiating triggers. Chronic pain independent of initiating triggers is maladaptive, offering no survival advantage, and very often no effective treatment is available.

Neuropathic pain can be classified as peripheral or central. Peripheral neuropathic pain is caused by injury or infection of peripheral sensory nerves, whereas central neuropathic pain is caused by damage to the CNS or/and the spinal cord. Both peripheral and central neuropathic pain can occur without obvious initial nerve damage.

In another embodiment of the invention, sabcomeline and the long-acting non-CNS penetrant muscarinic antagonist treat mood disorders. In another embodiment of the invention, sabcomeline and the long-acting non-CNS penetrant muscarinic antagonist treat movement disorders. In another embodiment of the invention, sabcomeline and the long-acting non-CNS penetrant muscarinic antagonist treat cognitive disorders, including enhancing cognitive function not associated with a specific pathology. In another embodiment of the invention, sabcomeline and the long-acting non-CNS penetrant muscarinic antagonist treat attention disorders. In another embodiment of the invention, sabcomeline and the long-acting non-CNS penetrant muscarinic antagonist treat pain.

In another aspect, sabcomeline and the long-acting non-CNS penetrant muscarinic antagonist can enhance attention, accelerate learning, and decrease fatigue due to both lack of sleep and circadian rhythm disturbances, such as jet lag. In another embodiment of the invention, sabcomeline and a long-acting non-CNS penetrant muscarinic antagonist treat addictive disorders.

As used herein, the term “mood disorder” includes major depressive disorder, dysthymia, recurrent brief depression, minor depression disorder, bipolar disorder, mania and anxiety.

As used herein, the term “movement disorders” includes, but is not limited to, Gilles de la Tourette's syndrome, Friederich's ataxia, Huntington's chorea, restless leg syndrome and other diseases or disorders whose symptoms include excessive movements, ticks and spasms.

As used herein, the term “attention disorders” refers to diseases or conditions marked by having an abnormal or decreased attention span. Diseases include, but are not limited to, attention deficit and hyperactivity disorder (ADHD), attention deficit disorder (ADD), Dubowitz Syndrome, FG Syndrome, Down's Syndrome, growth delay due to insulin-like growth factor I (IGF1) deficiency, hepatic encephalopathy syndrome, and Strauss Syndrome.

As used herein, the term “addictive disorders” refers to diseases or conditions marked by addiction or substance dependence. Such disorders are characterized by physical dependence, withdrawal and tolerance to a substance. Such substances include but are not limited to alcohol, cocaine, amphetamines, opioids, benzodiazepines, inhalants, nicotine, barbiturates, cocaine and cannabis. Addictive disorders also encompass behaviors that a patient does compulsively or continually despite clear negative consequences. For instance, ludomania (gambling addiction, or compulsive gambling) is recognized by those skilled in the art as being an addictive behavior that often has devastating consequences. In certain embodiments, the addictive behavior may be Internet Gaming Disorder (gaming addiction).

As used herein and where available, all conditions are defined according to their clinically-accepted criteria, which may include definitions in the Diagnostic and Statistical Manual of Mental Disorders, 5^{th} Edition, American Psychiatric Association, 2013 (DSM-V).

In one embodiment of the invention, sabcomeline combined with the long-acting non-CNS penetrant muscarinic antagonist can be used to treat an animal. In a further embodiment of the invention, the animal is a mammal. In an embodiment of the invention, the mammal is a human being.

In one embodiment of the invention, the long-acting non-CNS penetrant muscarinic antagonist decreases the side effects associated with sabcomeline. Such side effects include, but are not limited to, gastrointestinal (GI) side effects, cardiac side effects, excessive sweating, and excessive salivation. Use of the long-acting non-CNS penetrant muscarinic antagonist together with the sabcomeline allows the sabcomeline to be used clinically when the sabcomeline would not otherwise be used clinically due to its side effects. In another embodiment, use of the long-acting non-CNS penetrant muscarinic antagonist with the sabcomeline allows for the sabcomeline to achieve a higher maximum tolerated dose than sabcomeline would otherwise achieve. The use of a long-acting non-CNS muscarinic antagonist, such as glycopyrronium, or a pharmaceutically acceptable salt thereof, to decrease the side-effects associated with sabcomeline is preferable over the combination of sabcomeline with a shorter-acting antagonist such as trospium chloride, which will have weaker anti-secretory effects of a shorter duration and will not be able to maintain the mitigation of sabcomeline's potent peripheral M3 agonism for its duration.

In one embodiment of the invention, sabcomeline and the long-acting non-CNS penetrant muscarinic antagonist are administered to the subject as a single daily dose. In another embodiment of the invention, sabcomeline and the long-acting non-CNS penetrant muscarinic antagonist are administered to the subject once every 2 days, once every 3 days, once every 4 days, once every 5 days, once every 6 days, once every week, once every 8 days, once every 9 days, once every 10 days, once every 11 days, once every 12 days, once every 13 days, once every two weeks, once every 15 days, once every 16 days, once every 17 days, once every 18 days, once every 19 days, once every 20 days, once every 3 weeks, once every 22 days, once every 23 days, once every 24 days, once every 25 days, once every 26 days, once every 27 days, once every 4 weeks, once every 29 days, once every 30 days, once every month, once every 5 weeks, once every 6 weeks, once every 7 weeks, once every 8 weeks, once every 2 months, once every 9 weeks, once every 10 weeks, once every 11 weeks, once every 3 months, once every 12 weeks, once every 4 months, once every 5 months, once every 6 months, once every 9 months or once a year.

In one embodiment of the invention, sabcomeline and glycopyrronium, or a pharmaceutically acceptable salt thereof, are administered to the subject as a single daily dose. In another embodiment of the invention, sabcomeline and glycopyrronium, or a pharmaceutically acceptable salt thereof, are administered to the subject once every 2 days, once every 3 days, once every 4 days, once every 5 days, once every 6 days, once every week, once every 8 days, once every 9 days, once every 10 days, once every 11 days, once every 12 days, once every 13 days, once every two weeks, once every 15 days, once every 16 days, once every 17 days, once every 18 days, once every 19 days, once every 20 days, once every 3 weeks, once every 22 days, once every 23 days, once every 24 days, once every 25 days, once every 26 days, once every 27 days, once every 4 weeks, once every 29 days, once every 30 days, once every month, once every 5 weeks, once every 6 weeks, once every 7 weeks, once every 8 weeks, once every 2 months, once every 9 weeks, once every 10 weeks, once every 11 weeks, once every 3 months, once every 12 weeks, once every 4 months, once every 5 months, once every 6 months, once every 9 months or once a year.

In one embodiment of the invention, sabcomeline and glycopyrronium, or a pharmaceutically acceptable salt thereof, are administered to the subject as a single daily dose of no more than 20 mg of glycopyrronium, or a pharmaceutically acceptable salt thereof.

This invention will be better understood by reference to the Examples, which follow, but those skilled in the art will readily appreciate that the specific experiments detailed are only illustrative of the invention as described more fully in the claims which follow thereafter.

### EXAMPLES

### Example 1: Immediate Release Sabcomeline Hydrochloride and Glycopyrrolate Combination Oral Dosage Form

To make 100 kg of blend for tableting or encapsulation, at 100 μg sabcomeline/2 mg glycopyrrolate per 150 mg excipient, dissolve 67 g sabcomeline monohydrochloride and 1.34 kg of glycopyrrolate in 1 liter of water (i.e. 1% of water (1.067% of solution) on a weight basis). Slowly add this to 100 kg of a “direct compression” grade of lactose monohydrate in a high-shear mixer-granulator and blend together with 0.25 kg of magnesium stearate and 0.15 kg of colloidal silica, followed by pressing the mixture into tablets and film-coating.

### Example 2: Pilocarpine Induced Rat Salivation Model

2 mg/kg of pilocarpine dissolved in 5 mL/kg of physiological saline was orally administered to 3 cohorts of 6 anesthetized male Wistar (Han) rats (Janvier Labs). A fourth cohort of 6 animals received 5 mL/kg of intravenous physiological saline as a control. After 15 minutes, intravenous doses of glycopyrrolate and trospium chloride, each dissolved in 2.5 mL/kg of physiological saline and at doses analogous to their approved human doses (10 μg/kg and 100 μg/kg respectively) were separately administered to a cohort and the remaining 2 cohorts each received administrations of 2.5 mL/kg physiological saline intravenously. 15 minutes after this administration, the rats were anesthetized (Ketamine 75 mg/kg/Midazolam 0.5 mg/kg, i.p., 5 mL/kg body weight) and placed face down on a slightly inclined surface with the heads facing downwards to allow free flow of saliva and previously weighed cotton wool was introduced into each of the rat's mouths. Salivation responses were quantitated at 10-minute intervals for 30 minutes by collecting saliva on the cotton with the extracted cotton immediately weighed for the quantitation of saliva production (g). New fresh pieces were inserted at each time point.

The results of the assay are shown in Table 1 and demonstrated that anti-sialorrhea effect of trospium was both weaker and considerably shorter than that of glycopyrrolate. As the half-life of trospium in rats following intravenous administration is over 20 minutes, these results indicate that there is no clear PK/PD correlation in the anti-salivatory effect of trospium and is suggestive of a short dissociation time from submaxillary mucosa M3 receptors.

TABLE 1

| | wt % increase in saliva 20-30 minutes |
|---|---|
| Compound assayed | over 0-10 minutes |
| Glycopyrrolate | 38% |
| Trospium chloride | 211% |

### Example 3: M3 Receptor Binding Assay

The M3 binding of glycopyrrolate and trospium was assayed in a [3H]-NMS binding assay in 50 mM Tris-HCl buffered HEK293 cells at pH 7.4 (WuXi AppTec, HK). Both compounds were determined to have a Ki of 46 nM.

### Example 4: Sabcomeline Induced Rat Salivation Model

0.1 mg/kg of sabcomeline dissolved in 0.5% w/v methylcellulose in water was orally administered to 3 cohorts of 6 anesthetized male Wistar (Han) rats (Janvier Labs). A fourth cohort of 6 animals received 5 mL/kg of 5 mL/kg of 0.5% w/v methylcellulose in water as a control. After 15 minutes, intravenous doses of glycopyrrolate and trospium chloride, each dissolved in 2.5 mL/kg of physiological saline and at doses analogous to their approved human doses (10 μg/kg and 100 μg/kg respectively) were separately administered to a cohort and the remaining 2 cohorts each received administrations of 2.5 mL/kg physiological saline intravenously. 15 minutes after this administration, the rats were anesthetized (Ketamine 75 mg/kg/Midazolam 0.5 mg/kg, i.p., 5 mL/kg body weight) and placed face down on a slightly inclined surface with the heads facing downwards to allow free flow of saliva and previously weighed cotton wool was introduced into each of the rat's mouths. Salivation responses were quantitated at 10-minute intervals for 30 minutes by collecting saliva on the cotton with the extracted cotton immediately weighed for the quantitation of saliva production (g). New fresh pieces were inserted at each time point and upon completion of the study, blood samples were taken to assay for terminal drug plasma levels.

The results of the assay are shown in Table 2 and demonstrate that the anti-sialorrhea effect of trospium was both weaker and considerably shorter than that of glycopyrrolate.

TABLE 2

| | wt % increase in | wt % reduction in sabcomeline | |
|---|---|---|---|
| Compound | saliva 20-30 minutes | induced saliva | |
| assayed | over 0-10 minutes | 0-10 mins | 20-30 mins |
| Glycopyrrolate | 44% | 93% | 88% |
| Trospium | 211% | 88% | 53% |
| chloride | | | |

Terminal drug plasma levels of all drugs are shown in Table 3 and demonstrate that at the 30 minutes stage of the study, unbound sabcomeline levels were 170-fold higher than glycopyrrolate yet only 67-fold higher than trospium.

TABLE 3

| Compound assayed | Unbound drug (nM) | Unbound Sabcomeline (nM) |
|---|---|---|
| Glycopyrrolate | 0.116 | 19.8 |
| Trospium chloride | 0.33 | 22 |

These findings demonstrate that trospium's anti-sialorrhea effect of trospium was both weaker and considerably shorter than that of glycopyrrolate despite having comparable M3 binding and an almost 3-fold higher relative plasma exposure at the study's completion. The results indicate that there is no clear PK/PD correlation in the drug's anti-salivatory effect and are suggestive that glycopyrrolate's extended effects, which successfully counter sabcomeline's activity for the entire study duration, are actually due to its considerably longer dissociation time from submaxillary mucosa M3 receptors than trospium.

### Example 5: Antagonist Activity Assay

The antagonistic effect of glycopyrrolate and trospium on MIHEK/293 cell lines was investigated in a FLIPR assay (Wuxi Apptec Ltd., Hong Kong) as assayed against an EC₈₀ concentration of either arecaidine propargylester (reference agonist), sabcomeline or xanomeline. The results, as shown in Table 4 demonstrate that for the centrally and peripherally active M1 receptor, considerably higher than reference quantities of trospium are necessary to antagonize xanomeline, while only lower than reference quantities of glycopyrrolate are necessary to antagonize sabcomeline. From the results it could also be calculated that following the administration of the FDA approved 2 mg oral Robinul® glycopyrrolate tablet, peak circulating plasma exposures would be equivalent to the drug's EC₃₅, while following administration of the FDA approved 100/20 mg oral Cobenfy® xanomeline/trospium capsule, peak circulating plasma exposures would be equivalent to the drug's EC₈₇, suggesting that supratherapeutic quantities of trospium might be necessary to counter the peripheral agonist effects of xanomeline.

TABLE 4

| | Glycopyrrolate | Trospium |
|---|---|---|
| % IC₅₀ sabcomeline/IC₅₀ reference | 62% | 100% |
| % IC₅₀ xanomeline/IC₅₀ reference | — | 167% |

### Example 6: Dog PK Study

The safety and pharmacokinetic profile of sabcomeline and glycopyrrolate were tested in an oral gavage administration study in female beagle dogs. 150 μg/kg of sabcomeline HCl was orally administered by gavage tube either alone or together with 150 μg/kg of glycopyrrolate HBr to cohorts of 3 female beagle dogs each weighing no more than 11 kg and the dogs observed cage-side for clinical observations of general health and appearance by a qualified veterinarian or veterinarian technician. Adverse events (AEs) were defined to include both vomiting and the production of liquid feces. Blood samples were also taken at regular predetermined intervals and analyzed to identify their respective glycopyrrolate content.

Three dogs received sabcomeline alone and a total of fifteen received the combination of both sabcomeline and glycopyrrolate. Each of the three dogs that received sabcomeline alone presented with at least 2 different adverse events, while of the fifteen dogs that received the combination of both sabcomeline and glycopyrrolate, 10 had no identifiable adverse events, 2 dogs presented with 1 adverse event and 3 with at least 2 different adverse events, thereby indicating that the glycopyrrolate was successful in mitigating the gastrointestinal effects of sabcomeline. Analysis of the blood plasma, as taken from nine of the fifteen dogs administered the combination, identified that peak circulating plasma exposures of glycopyrrolate were equivalent to the drug's ECs, thereby indicating that glycopyrrolate can successfully mitigate the peripheral adverse events of sabcomeline even potentially at sub-therapeutic doses.

The invention described herein may be practiced in the absence of any element or limitation which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method of treating a disorder ameliorated by activating muscarinic receptors in a subject in need thereof, comprising administering to a patient in need thereof an effective amount of a fixed dose combination formulation comprising between about 25 μg and about 60 mg of sabcomeline or a pharmaceutically acceptable salt thereof, between about 0.01 mg and about 600 mg of a long-acting non-Central Nervous System (CNS) penetrant muscarinic antagonist, and at least one pharmaceutically acceptable excipient, and wherein the non-CNS penetrant muscarinic antagonist is present in an amount sufficient to decrease at least one side effect associated with the sabcomeline present in the fixed dose combination formulation in the subject.

2. The method of claim 1, wherein the disorder is selected from the group consisting of schizophrenia, Alzheimer's disease, Parkinson's disease, depression, movement disorders, pain, drug addiction, tauopathy, and synucleinopathy.

3. The method of claim 1, wherein the at least one side effect is selected from the group consisting of gastrointestinal side effects, cardiac side effects, excessive sweating, excessive salivation, and combinations thereof.

4. The method of claim 1, wherein sabcomeline or the pharmaceutically acceptable salt thereof, is present in the fixed dose combination formulation in an amount higher than a dose of sabcomeline that could be tolerated in the absence of the long-acting non-CNS penetrant muscarinic antagonist.

5. The method of claim 1, wherein the long-acting non-CNS penetrant muscarinic antagonist is present in an amount sufficient to partially or completely inhibit sabcomeline's peripheral M3 agonism.

6. The method of claim 1, wherein the fixed dose combination formulation is administered once daily, once weekly, once monthly, once every three months, once every six months, once every 9 months, or once a year.

7. The method of claim 1, wherein the long-acting non-CNS penetrant muscarinic antagonist is glycopyrronium or a pharmaceutically acceptable salt thereof.

8. The method of claim 7, wherein the long-acting non-CNS penetrant muscarinic antagonist is glycopyrrolate.

9. The method of claim 7, wherein the fixed dose combination formulation comprises an effective amount of glycopyrronium, or an equivalent amount of the pharmaceutically acceptable salt thereof, that does not exceed 20 mg.

10. The method of claim 7, wherein the fixed dose combination formulation comprises an effective amount of glycopyrronium, or an equivalent amount of the pharmaceutically acceptable salt thereof, that does not exceed 10 mg.

11. The method of claim 1, wherein the fixed dose combination formulation is administered once daily.

12. The method of claim 11, wherein the fixed dose combination formulation is administered orally.

13. The method of claim 12, wherein the fixed dose combination formulation is administered orally as a tablet, troche, liquid, emulsion, suspension, drop, capsule, caplet, or gel cap.

14. The method of claim 1, wherein the fixed dose combination formulation is administered parenterally, optically, transdermally, nasally, ocularly, rectally, sublingually, via inhalation, or vaginally.

15. A fixed dose pharmaceutical composition comprising between about 25 μg and about 60 mg of sabcomeline, or a pharmaceutically acceptable salt thereof, and between about 0.01 mg and about 600 mg of a long-acting non-Central Nervous System (CNS) penetrant muscarinic antagonist, and at least one pharmaceutically acceptable excipient, and wherein the long acting non-CNS penetrant muscarinic antagonist is present in an amount sufficient to decrease at least one side effect associated with the sabcomeline or the equivalent amount of the pharmaceutically acceptable salt thereof when the fixed dose combination formulation is administered to a subject in need thereof.

16. The fixed dose pharmaceutical composition of claim 15, wherein the pharmaceutical composition comprises an oral dosage form comprising between about 25 μg and about 1 mg of sabcomeline, or a pharmaceutically acceptable salt thereof.

17. The fixed dose pharmaceutical composition of claim 16, wherein the pharmaceutical composition comprises between about 0.01 mg and about 20 mg of the long-acting non-CNS penetrant muscarinic antagonist.

18. The fixed dose pharmaceutical composition of claim 15, wherein the pharmaceutical composition comprises a parenteral dosage form comprising between about 25 μg and about 60 mg of sabcomeline, or a pharmaceutically acceptable salt thereof.

19. The fixed dose pharmaceutical composition of claim 18, wherein the pharmaceutical composition comprises between about 0.01 mg and about 600 mg of the long-acting non-CNS penetrant muscarinic antagonist.

20. The fixed dose pharmaceutical composition of claim 15, wherein the pharmaceutical composition provides a daily dose of between about 25 μg and about 1 mg of sabcomeline, or a pharmaceutically acceptable salt thereof.

21. The fixed dose pharmaceutical composition of claim 15, wherein the pharmaceutical composition provides a daily dose of the long-acting non-CNS penetrant muscarinic antagonist of between about 0.01 mg and about 10 mg.

22. The fixed dose pharmaceutical composition of claim 21, wherein the pharmaceutical composition provides a daily dose of the long-acting non-CNS penetrant muscarinic antagonist of between about 0.01 mg and about 5 mg.

23. The fixed dose pharmaceutical composition of claim 22, wherein the pharmaceutical composition provides a daily dose of the long-acting non-CNS penetrant muscarinic antagonist of between about 0.01 mg and about 2 mg.

24. The fixed dose pharmaceutical composition of claim 15, wherein the long-acting non-CNS penetrant muscarinic antagonist is selected from the group consisting of aclidinium, glycopyrronium, tiotropium and pharmaceutically acceptable salts thereof.

25. The fixed dose pharmaceutical composition of claim 24, wherein the long-acting non-CNS penetrant muscarinic antagonist comprises glycopyrronium or a pharmaceutically acceptable salt thereof.

26. The fixed dose pharmaceutical composition of claim 25, wherein the long-acting non-CNS penetrant muscarinic antagonist comprises glycopyrrolate.

27. The fixed does combination formulation of claim 15, wherein sabcomeline or the equivalent amount of the pharmaceutically acceptable salt thereof, is present in the fixed dose combination formulation in an amount higher than a dose of sabcomeline that could be tolerated in a subject in the absence of the long-acting non-CNS penetrant muscarinic antagonist.

28. The fixed dose combination formulation of claim 15, wherein the long-acting non-CNS penetrant muscarinic antagonist is present in the formulation in an amount sufficient to partially or completely inhibit sabcomeline's peripheral M3 agonism when the fixed dose combination formulation is administered to a subject in need thereof.

29. A fixed dose pharmaceutical composition comprising 25 μg and about 1 mg of sabcomeline, or a pharmaceutically acceptable salt thereof, and glycopyrronium or a pharmaceutically acceptable salt thereof, wherein the pharmaceutical composition provides a daily dose of glycopyrronium or a pharmaceutically acceptable salt thereof of between about 0.01 mg and about 10 mg.

30. An oral dosage form comprising 25 μg and about 1 mg of sabcomeline, or a pharmaceutically acceptable salt thereof and between about 0.01 mg and about 10 mg of glycopyrronium or a pharmaceutically acceptable salt thereof and wherein administration of the oral dosage form provides for a daily dose of no more than about 10 mg of glycopyrronium or a pharmaceutically acceptable salt thereof.
